(19) United States
(12) Patent Application Publication
(10) Pub. No.: US 2006/0235385 A1
(43) Pub. Date: Oct. 19, 2006

(54) Low profile polyaxial screw

(76) Inventor: Dale Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/095,625

(22) Filed: Mar. 31, 2005

(51) Int. Cl. A61F 2/30

(52) U.S. Cl. 606/61

(57) ABSTRACT

A bone anchor assembly includes a bone anchor having a distal shaft configured to engage bone and a hollow hemi-spherical proximal head defined by a convex outer surface and a concave inner surface, a receiving member for receiving a spinal fixation element and for engaging the head of the bone anchor, and a compression member positionable in the receiving member. The compression member has an upper portion configured to seat the spinal fixation element and a lower portion configured to engage the concave inner surface of the anchor head.

### BACKGROUND

Spinal fixation systems may be used in surgery to align, adjust and/or fix portions of the spinal column, i.e., vertebrae, in a desired spatial relationship relative to each other. Many spinal fixation systems employ a spinal rod for supporting the spine and for properly positioning components of the spine for various treatment purposes. Vertebral anchors, comprising pins, bolts, screws, and hooks, engage the vertebrae and connect the supporting rod to different vertebrae. The size, length and shape of the cylindrical rod depend on the size, number and position of the vertebrae to be held in a desired spatial relationship relative to each other by the apparatus.

Spinal fixation elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal fixation element-receiving element, which, in spinal rod applications, is usually in the form of a U-shaped slot formed in the head portion for receiving the rod. A set-screw, plug, cap or similar type of closure mechanism is used to lock the rod into the rod-receiving portion of the pedicle screw. In use, the shank portion of each screw is then threaded into a vertebra, and once properly positioned, a fixation rod is seated through the rod-receiving portion of each screw. The rod is locked into place by tightening a cap or similar type of closure mechanism to securely interconnect each screw and the fixation rod. Other anchoring devices also include hooks and other types of bone screws.

Polyaxial pedicle screws have been designed to allow angulation of one portion of the screw relative to another portion of the screw and the spinal fixation element coupled to one portion of the screw. For example, polyaxial pedicle screws allow for a shaft portion to pivot relative to a rod-receiving portion in all directions about a 360° arc around the rod-receiving portion. Polyaxial screws may be useful for positioning bone anchors on adjacent vertebrae, when the close proximity of adjacent vertebrae can result in interference between the bone anchors. Polyaxial screws allow for pivoting of the screws in any direction out of alignment with each other to avoid such interference.

An example of such a polyaxial pedicle screw assembly is described in detail in U.S. Patent Application Publication Number US 2004/0186473 entitled “Spinal Fixation Devices of Improved Strength and Rigidity”, U.S. Patent Application Publication Number US 2004/0181224 entitled “Anchoring Element for Use in Spine or Bone Surgery, Methods for Use and Production Thereof” and U.S. Patent Application Publication Number US 2003/0100896, entitled “Element With a Shank and a Holding Element Connected to It for Connecting to a Rod”, the contents of which are herein incorporated by reference.

Polyaxial and multi-axial screws, which allow the screw shank to pivot in all directions about the head portion, can have high profiles to accommodate the polyaxial mechanism and to provide the strength needed to secure the spinal rod to the vertebral body. However, high profile polyaxial screws are not desirable in areas where there is little distance between the vertebral body and the patient's skin such as in the posterior spine.

### SUMMARY

Disclosed herein are bone screw assemblies having a reduced profile that provide for polyaxial movement between an anchor portion and a rod-receiving portion of the bone screw assembly. The bone screw assemblies disclosed herein allow the anchor portion to pivot about the rod-receiving portion in one or more directions.

In accordance with one aspect, an exemplary embodiment of a bone anchor assembly may comprise a bone anchor having a distal shaft configured to engage bone and a hollow hemi-spherical proximal head defined by a convex outer surface and a concave inner surface, a receiving member for receiving a spinal fixation element and for engaging the head of the bone anchor, and a compression member positionable in the receiving member. The compression member, in the exemplary embodiment may have an upper portion configured to seat the spinal fixation element and a lower portion configured to engage the concave inner surface of the anchor head.

According to another exemplary embodiment, a bone anchor assembly may comprise a bone anchor having a distal shaft configured to engage bone and a hollow hemi-spherical proximal head defined by a convex outer surface and a concave inner surface, a receiving member for receiving a spinal fixation element and the head of the bone anchor, and a compression member positionable in the receiving member. In the exemplary embodiment, the convex outer surface and the concave inner surface may be spaced apart to form a wall having a thickness and the thickness of the wall may be approximately less than or equal to 33% of a radius of the convex outer surface. The compression member, in the exemplary embodiment, may have an upper portion including a groove to seat the spinal fixation element and a lower portion having a convex shape having a radius approximating a radius of the concave inner surface of the head of the bone anchor.

According with a further exemplary embodiment, a kit may comprise a spinal fixation element, a bone anchor assembly, and an instrument configured to engage a drive feature on the head of the bone anchor of the bone anchor assembly. The bone anchor assembly, in the exemplary embodiment, may comprise a bone anchor having a distal anchoring shaft and a proximal hollow hemispherical head defined by an outer convex surface and an inner concave surface, a rod-receiving member for receiving the spinal fixation element and the head of the bone anchor, and a compression member positionable within the rod-receiving member and configured to engage the hollow head of the bone anchor. The head of the bone anchor, in the exemplary embodiment, may have a drive feature positioned between the outer convex surface and the inner concave surface.

### BRIEF DESCRIPTION OF THE DRAWINGS

The foregoing and other objects, features and advantages of the bone anchor assemblies disclosed herein will be apparent from the following description and apparent from the accompanying drawings, in which like reference characters refer to the same parts throughout the different views. The drawings illustrate principles of the invention and, although not to scale, show relative dimensions.

FIG. 1 is a side view in cross-section of an exemplary embodiment of a low profile bone screw assembly;

FIG. 2 is a perspective view of the anchor portion of the bone screw assembly of FIG. 1;

FIG. 3A is a top view of the upper portion of the compression member of the bone screw assembly of FIG. 1;

FIG. 3B is a bottom view of the lower portion of the compression member of the bone screw assembly of FIG. 1;

FIG. 4 is a perspective view of the rod-receiving member of the bone screw assembly of FIG. 1;

FIG. 5A is a perspective view of the anchor member of another embodiment of a bone anchor assembly;

FIG. 5B is a perspective view of a compression member for use with the anchor member of FIG. 5A, illustrating access channels for engaging the drive feature on the anchor member;

FIG. 5C is a perspective view of an alternate embodiment of a drive feature for the anchor member illustrated in FIG. 5A; and

FIG. 6 is a perspective view of an exemplary instrument for engaging the drive feature of the bone anchor of FIG. 5A.

### DETAILED DESCRIPTION OF EXEMPLARY EMBODIMENTS

Certain exemplary embodiments will now be described to provide an overall understanding of the principles of the structure, function, manufacture, and use of the bone anchor assemblies and methods disclosed herein. One or more examples of these embodiments are illustrated in the accompanying drawings. Those of ordinary skill in the art will understand that the bone anchor assemblies and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

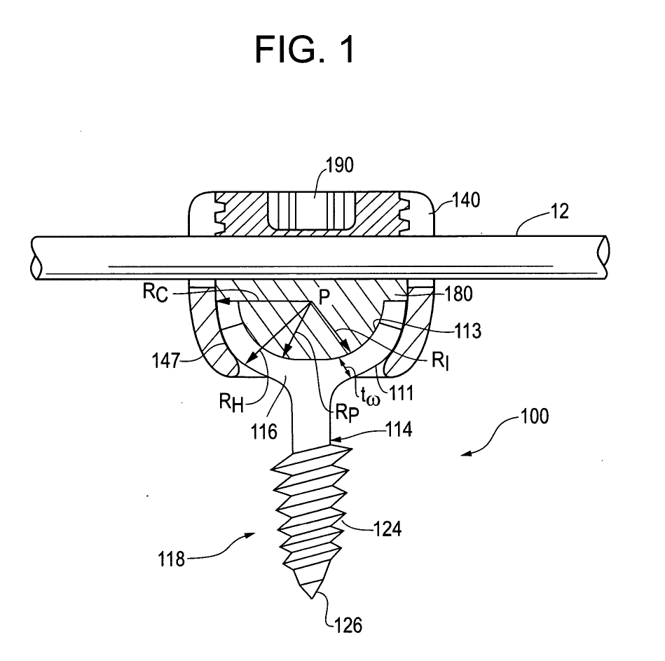

During spinal surgeries, polyaxial and multi-axial screw assemblies may be used to fix spinal rods, cables or plates to the vertebral bodies at the pedicle. A polyaxial screw assembly having a low profile would be beneficial to the patient in reducing tissue irritation. Different exemplary embodiments of a low profile polyaxial screw assembly are illustrated in FIGS. 1-5. The illustrated assemblies allow for angulation of the anchor portion relative to a head portion in one or more, (including all) planes, while minimizing the overall height of the assembly.

The exemplary bone screw assemblies may be employed to engage one or more spinal fixation elements to bone. For example, a bone screw assembly may be employed to fix a spinal plate, rod, and/or cable to a vertebra of the spine. Although the exemplary bone screw assemblies described below are designed primarily for use in spinal applications, and specifically the pedicle region of a vertebra, one skilled in the art will appreciate that the structure, features and principles of the exemplary bone screw assemblies, as well as the other exemplary embodiments described below, may be employed to couple any type of orthopedic implant to any type of bone or tissue.

An exemplary embodiment of a low profile polyaxial bone screw assembly 100, as illustrated in FIGS. 1-4, may include a bone anchor 114 having a distal shaft 118 configured to engage bone and a proximal head 116, a receiver member 140 for receiving a spinal fixation element, such as a spinal rod 12, and a compression member 180 positionable in the receiving member 140 and configured to engage the head 116 of the bone anchor 114 and provide a seat for receiving the spinal fixation element. In the illustrated embodiment, the bone anchor assembly 100 is polyaxial, e.g., the head 116 of the bone anchor 114 is adjustable relative to the receiver member 140 to allow for polyaxial movement between the bone anchor 114 and rod-receiving portion 140.

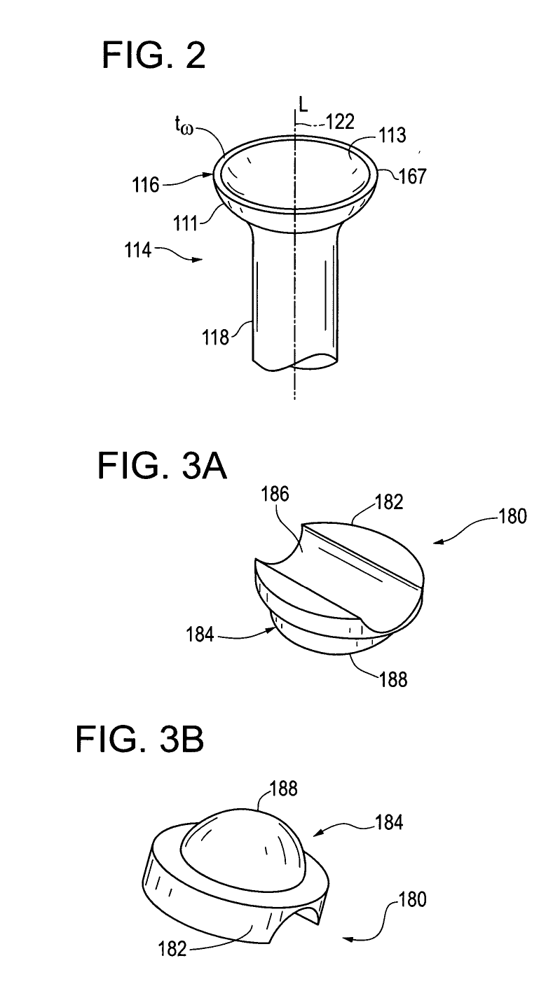

The bone anchor 114 has a proximal end and a distal end and a longitudinal axis 122 extending therebetween. The proximal head 116 is provided at the proximal end of the bone anchor 114. The exemplary anchor head 116 is hollow and has a generally hemi-spherical shape defined by an outer convex surface 111 and an inner concave surface 113. In the exemplary embodiment, the outer convex surface 111 and the inner concave surface 113 are generally spherical in shape. The convex outer surface has a radius R_{H} as shown in FIG. 2. The inner convex surface has a radius R_{I}. In the illustrated embodiment, the outer concave surface 111 and the inner convex surface 113 have a common center point P. The outer concave surface 111 and the inner convex surface 113 are spaced apart a distance to form a wall 167 having a thickness T_{w} The thickness T_{w} of the wall 167 is a percentage of the radius R_{H} of the outer convex surface 113 of the head 116. For example, in one exemplary embodiment, the thickness T_{w} of the wall 167 is less than or equal to approximately 40% of a radius of the outer convex surface 113 of the head 116. In another exemplary embodiment, the thickness T_{w} of the wall 167 is less than or equal to approximately 33% of a radius of the outer convex surface 113 of the head 116. In another exemplary embodiment, the thickness T_{w} of the wall 167 is less than or equal to approximately 20% of a radius of the outer convex surface 113 of the head 116. The outer convex surface 113 of the head 116 may also have texturing such as threads, knurling, or bead blasting to facilitate engagement with the receiver member 140.

The distal shaft 118 may include one or more bone engagement mechanisms to facilitate gripping engagement of the bone anchor to bone. In the illustrated embodiment, the distal shaft 118 includes an external thread 124 extending along at least a portion of the shaft for engaging bone. In the illustrated embodiment, the external thread 124 is a single lead thread that extends from a distal tip 126 of the shaft to the anchor head 116, though one skilled in the art will recognize that the external thread may extend along any selected portion of the shaft and have any suitable number of leads. Other suitable bone engagement mechanisms include, but are not limited to, one or more annular ridges, multiple threads, dual lead threads, variable pitched threads and/or any conventional bone engagement mechanism.

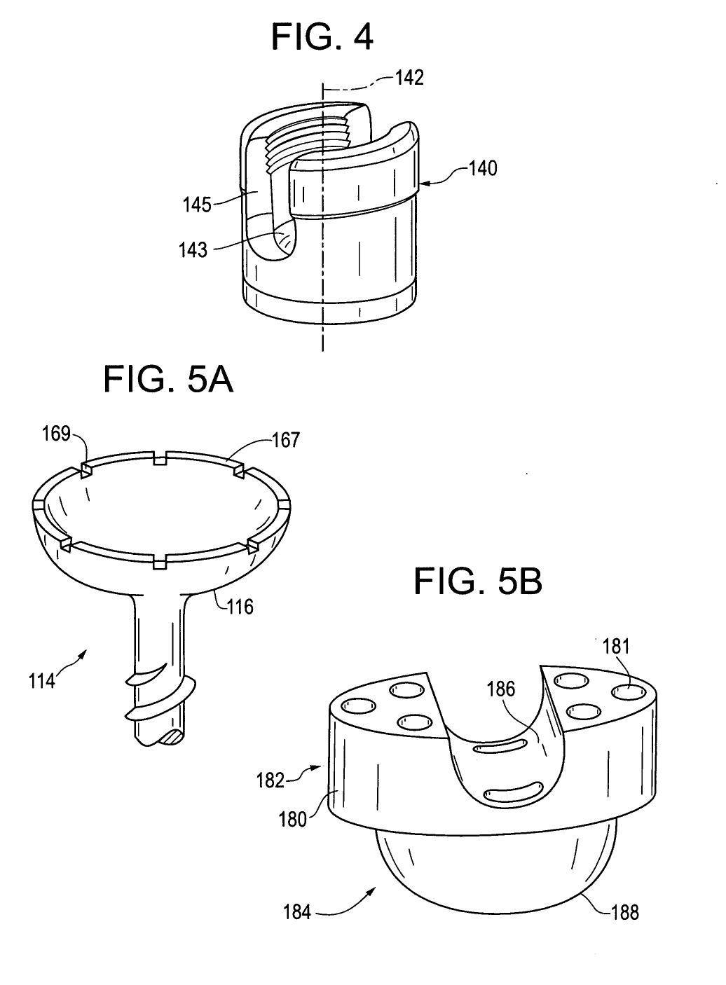

The rod-receiving member 140 shown in FIG. 4, has an upper and lower portion, a U-shaped channel 145 for receiving a spinal fixation element, such as a spinal rod 12, and an axial bore 143 extending therethrough. The lower portion of the rod-receiving member 140 has a complementary concave or spherical shape 147 to the outer convex surface 113 of the anchor head 116. The axial bore 143 has a diameter larger than the diameter of the shaft 118 of the bone anchor 114, but smaller than the extent, e.g., the diameter, of the head 116 of the bone anchor 114. This relationship allows for a top-loading screw assembly where the shaft 118 of the bone anchor 114 may be inserted through the axial bore 143 at the top (e.g., proximal end) of the receiving member 140. In alternative embodiments, the shaft 118 of the bone anchor 114 may be inserted from the bottom (e.g., distal end) of the receiving member 140 and captured by a retaining mechanism, such as a ring or clip, within the distal portion of the receiving member 140. Such embodiments are generally referred to as bottom-loading screw assemblies. In a neutral position, the longitudinal axis 122 of the bone anchor 114 is aligned with a longitudinal axis 142 extending through the rod-receiving member 140. In the exemplary polyaxial screw assembly, the shaft 118 of the bone anchor 114 is pivotable relative to the rod-receiving member 140 such that the shaft 118 is adjustable in one or more planes relative to the receiver member 140.

The U-shaped channel 145 of the receiving member 140 of the exemplary bone screw assembly 100 may be sized and shaped to receive a spinal rod 12 or another suitable spinal fixation element. The exemplary spinal rod 12 may be seated within the channel 145 by aligning the spinal rod 12 and the channel 145 and advancing the spinal rod through the top into the channel 145. The configuration of the channel 145 may be varied to accommodate any suitable spinal fixation element. A suitable configuration for the receiving member 140 is described in the U.S. Patent Application Publication Numbers US 2004/0186473, US 2004/0181224 and US 2003/0100896, the contents of which are herein incorporated by reference.

Continuing to refer to FIGS. 1-4, the compression member 180 of the exemplary embodiment includes an upper (proximal) portion 182 configured to seat a spinal fixation element such as spinal rod 12 and a lower (distal) portion 184 configured to engage the proximal head 116 of the bone anchor 114. In the exemplary embodiment, the compression member 180 may be positioned in the lower (distal) portion of the rod-receiving member 140, within the axial bore 143, proximal to and in engagement with the anchor head 116. The upper portion 182 of the compression member 180, as illustrated in FIG. 3A, includes a groove 186 formed in the proximal surface of the upper portion 182. The groove 186 defines a seat for a spinal rod or other spinal fixation element. The groove 186 has a generally arcuate cross-section having a curvature that may approximate the curvature of the exemplary spinal rod to be received therein. The opposed lower portion 184 of the compression member 180 may have a convex outer surface 188 for engaging the concave inner surface 113 of the anchor head 116. The convex outer surface 188 of the lower portion 184 has a radius Rₚ, as illustrated in FIG. 1, that approximates, and is preferable equal to, the radius R_{I} of the inner concave surface 113 of the anchor head 116. In the illustrated embodiment, the convex outer surface 188 of the lower portion 184 of the compression member 180 and the concave inner surface 113 of the proximal head 116 of the bone anchor 114 have a common center point P. Providing a common center point for the engagement surfaces and increasing the surface area of contact between the outer convex surface 188 of the lower portion 184 of the compression member, as described below, increases the stability of the bone anchor assembly when a locking element is engaged to fix the position of the spinal fixation element and the bone anchor relative to the bone anchor assembly.

The upper portion 182 of the compression member 180 of the exemplary bone anchor assembly 100 is generally disc-shaped having a circular cross-section or other cross section preferably corresponding to the axial bore 143 of the receiving member 140. The upper portion 182 may have a radius R_{c} extending from a center point of the upper portion 182 to the outer radial edge of the compression member 180. The radius Rₚ of the convex outer surface 188 of the lower portion 184 of the compression member 180 may be a percentage of the radius R_{c} of the upper portion 182 of the compression member 180. In one embodiment, for example, the radius Rₚ of the convex outer surface 188 of the lower portion 184 of the compression member 180 is greater than or equal to approximately 85% of the radius R_{c} of the upper portion 182 of the compression member 180. In another embodiment, for example, the radius Rₚ of the convex outer surface 188 of the lower portion 184 of the compression member 180 is greater than or equal to approximately 75% of the radius R_{c} of the upper portion 182 of the compression member 180. In another embodiment, for example, the radius Rₚ of the convex outer surface 188 of the lower portion 184 of the compression member 180 is greater than or equal to approximately 65% of the radius R_{c} of the upper portion 182 of the compression member 180.

The exemplary bone anchor assembly may further include a locking element to secure the fixation element relative to the receiving member and the bone anchor. In the exemplary embodiment, for example, a locking element 190 may fix the spinal rod 12 within the U-shaped channel 145 of the receiving member 140 and fix the position of the anchor head 116 with respect to the receiver member 140. In particular, the locking element 190 engages the spinal rod 12 and seats the rod 12 within the groove 186 of the compression member 180 and advances the lower portion 184 of the compression member 180 into fixed engagement with the proximal head 116 of the bone anchor 114. The locking element 190 can be in a threaded set screw, as in the illustrated embodiment, a twist-in cap, an external locking nut, a combination thereof or any other locking element known to one skilled in the art.

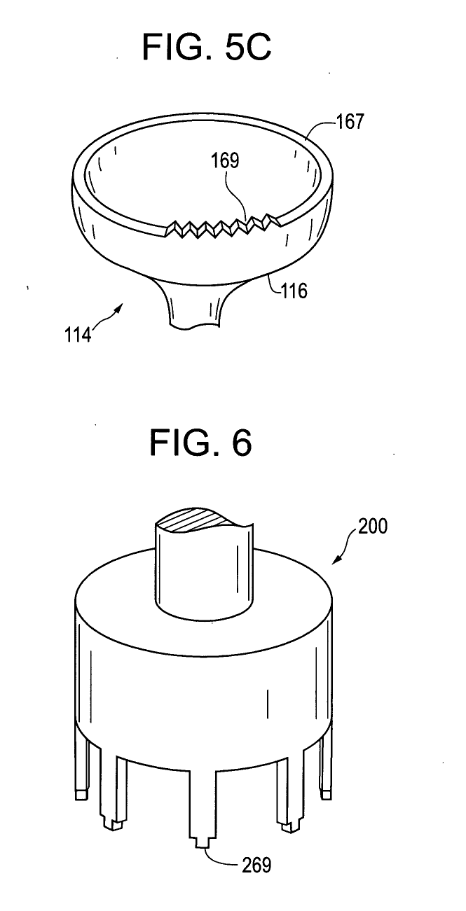

In the exemplary embodiment, the proximal head 116 of the bone anchor 114 may include a drive feature 169 positioned on the wall 167 formed between the inner concave surface 113 and the outer convex surfaces 111 of the proximal head 116. The drive feature 169 may be adapted to mate with an instrument to drive the screw assembly into bone. As shown in FIG. 5A, the drive feature 169 may have various shaped notches positioned around the wall 167 that mate with the complementary shaped notches on the tip of an instrument to form an interlocking connection between the instrument and the shank for transmitting torque. An example of a driver instrument 200 having mating shapes 269 is shown in FIG. 6. An alternate embodiment of a drive feature 169 has a sawtooth design around the wall as shown in FIG. 5C. One skilled in the art will recognize that any other type of drive feature capable of transmitting torque may be used.

As shown in FIG. 1, the compression member 180 is positioned within the receiving member 140 between the spinal fixation element, illustrated as a rod 12, and the anchor head 116 when the bone screw assembly is assembled. When the locking element 190 is engaged, the spinal rod 12 engages the compression member 180 which engages the anchor head 116 to anchor the rod to the bone and prevent further polyaxial movement between the anchor shaft and the receiver member. The compression member 180 may be swaged or threaded into position within the receiving member 140. The compression member 180 may further have at least one opening 181 or channel for allowing advancement of an instrument to the drive feature on the wall of the anchor head during implantation of the bone screw assembly. An exemplary embodiment of a compression member 180 including an opening 181 providing access to a drive feature 169 provided on the proximal head 116 of the bone anchor 114 is shown in FIG. 5B.

After pivoting the bone anchor portion 116 about an axis relative to the receiving portion 140, a user can lock the orientation of the bone anchor 114 relative to the receiving portion 140 by inserting the locking element 190. The locking element 190 secures a spinal rod 12 or other suitably configured spinal fixation element within the channel 145 of the receiving member 140 and locks the anchor head 116 in the selected orientation within and relative to the receiving member 140. In the illustrative embodiment, advancing the locking element 190 into engagement with the spinal rod 12 in the channel 145 seats the spinal rod 12 in the seat 186 of the compression member 180. The compression member 180 compresses against the inner concave surface 113 of the anchor head 116 to lock the bone anchor 114 in the selected orientation.

While the illustrative embodiment is a top-loading screw, one skilled in the art will recognize that the present invention encompasses a bottom-loading screw as well. A top-loading screw is assembled by inserting the shaft in a distal direction through the bottom opening, so that the anchor head is retained within a cavity in the receiving member. A bottom-loading screw is assembled by inserting the anchor head in a proximal direction through the bottom opening, and activating a securing means to prevent the anchor head from passing through the opening.

Another embodiment of the invention includes a bone anchor system. The system has at least one bone anchor having an anchor head 116, a shaft 118, a rod-receiving member 140 and a compression member 180. Also included in the system is an instrument 200 for driving the bone anchor assembly, a spinal fixation element 12, and a locking element 190 for securing the fixation element to the bone anchor. The individual components are as described above.

The components of the bone anchor assemblies described above may be manufactured from any suitable biocompatible material, including, but not limited to, metals and metal alloys such as titanium and stainless steel, polymers, ceramics, and/or composites thereof. The components may be manufactured from the same or different materials though manufacturing processes known in the art.

While the bone anchor assemblies and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

It is also to be understood that the following claims are to cover all generic and specific features of the invention described herein, and all statements of the scope of the invention, which, as a matter of language, might be said to fall therebetween.

1. A bone anchor assembly comprising:
a bone anchor having a distal shaft configured to engage bone and a hollow hemi-spherical proximal head defined by a convex outer surface and a concave inner surface;
a receiving member for receiving a spinal fixation element and for engaging the head of the bone anchor; and
a compression member positionable in the receiving member, the compression member having an upper portion configured to seat the spinal fixation element and a lower portion configured to engage the concave inner surface of the anchor head.

2. The bone anchor assembly of claim 1, further comprising a locking element for securing the spinal fixation element within the receiving member.

3. The bone anchor assembly of claim 2, wherein the locking element is a set screw.

4. The bone anchor assembly of claim 2, wherein the locking element is a twist in cap.

5. The bone anchor assembly of claim 1, wherein the head of the bone anchor includes a drive feature positioned between the convex outer surface and the concave inner surface.

6. The bone anchor assembly of claim 5, wherein the drive feature comprises a sawtooth configuration.

7. The bone anchor assembly of claim 1, wherein the compression member has at least one opening for accessing a drive feature on the head on the bone anchor.

8. The bone anchor assembly of claim 7, wherein the compression member includes an anti-rotation feature to prevent the compression member from rotating with respect to the receiver member.

9. The bone anchor assembly of claim 1, wherein the convex outer surface of the head has texturing.

10. The bone anchor assembly of claim 1, wherein the concave inner surface of the head and the lower portion of the compression member have a common center point.

11. A bone anchor assembly of claim 1, wherein the upper portion of the compression member is generally disc shaped and has a groove formed in the proximal surface thereof for seating the spinal fixation element.

12. The bone anchor assembly of claim 11, wherein the lower portion of the compression member has a convex shape having a radius approximating a radius of the concave inner surface of the head of the bone anchor.

13. The bone anchor assembly of claim 12, wherein the radius of the lower portion of the compression member is greater than or equal to approximately 75% of a radius of the upper portion of the compression member.

14. The bone anchor assembly of claim 12, wherein the radius of the lower portion of the compression member is greater than or equal to approximately 66% of a radius of the upper portion of the compression member.

15. The bone anchor assembly of claim 1, wherein the convex outer surface and the concave inner surface of the head of the bone anchor are spaced apart to form a wall having a thickness.

16. The bone anchor assembly of claim 15, wherein the thickness of the wall is less than or equal to approximately 33% of a radius of the convex outer surface of the head of the bone anchor.

17. The bone anchor assembly of claim 15, wherein the thickness of the wall is less than or equal to approximately 20% of a radius of the convex outer surface of the head of the bone anchor.

18. A kit comprising:
a spinal fixation element;
a bone anchor assembly comprising
bone anchor having a distal anchoring shaft and a proximal hollow hemispherical head defined by an outer convex surface and an inner concave surface, the head having a drive feature positioned between the outer convex surface and the inner concave surface,
a rod-receiving member for receiving the spinal fixation element and the head of the bone anchor,
a compression member positionable within the rod-receiving member and configured to engage the hollow head of the bone anchor, and
a locking mechanism for selective locking the spinal fixation element relative to the bone anchor; and
an instrument configured to engage the drive feature on the head.

19. A bone anchor assembly, comprising:
a bone anchor having a distal shaft configured to engage bone and a hollow hemi-spherical proximal head defined by a convex outer surface and a concave inner surface, the convex outer surface and the concave inner surface being spaced apart to form a wall having a thickness, the thickness of the wall being approximately less than or equal to 33% of a radius of the convex outer surface;
a receiving member for receiving a spinal fixation element and the head of the bone anchor; and
a compression member positionable in the receiving member, the compression member having an upper portion including a groove to seat the spinal fixation element and a lower portion having a convex shape having a radius approximating a radius of the concave inner surface of the head of the bone anchor.
